(19) United States
(12) Patent Application Publication
(10) Pub. No.: US 2008/0295203 A1
(43) Pub. Date: Nov. 27, 2008

(54) EXPRESSION OF AN ANTIMICROBIAL PEPTIDE VIA THE PLASTID GENOME TO CONTROL PHYTOPATHOGENIC BACTERIA

(76) Inventor: Henry Daniell, Winter Park, FL (US)

Correspondence Address: Beusse Wolter Sanks Mora & Maire, 390 N. ORANGE AVENUE, SUITE 2500, ORLANDO, FL, 32801, US

(21) Appl. No.: 12/042,607

(22) Filed: Mar. 5, 2008

Related U.S. Application Data
(63) Continuation of application No. 11/914,469, filed on Jan. 30, 2008.
Application No. 11/914,469 is a continuation of application No. 10/671,822, filed on Jan. 20, 2004, now abandoned.
(60) Provisional application No. 60/685,733, filed on May 27, 2005.

(51) Int. Cl. C12N 15/82; A01H 5/00

(52) U.S. Cl. 800/279; 435/320.1; 800/301

(57) ABSTRACT

This invention provides a novel method to confer disease resistance to plants. Plant plastids are transformed using a plastid vector which contains heterologous DNA sequences coding for a cytotoxic antimicrobial peptide. Transgenic plants are capable of fighting off phytopathogenic bacterial infection.

### CROSS-REFERENCE TO RELATED APPLICATION(S)

The present application is a continuation of U.S. application Ser. No. 11/914,469 filed Nov. 15, 2007, which is a U.S. National stage filing of PCT/US06/21020 filed May 30, 2006 which claims priority to U.S. Ser. No. 60/685,733, now lapsed.

### FIELD OF INVENTION

This application pertains to the field of genetic engineering of plant genomes, particularly plastids, and to methods of and engineered plants that express antimicrobial peptides that lead to and result in phytopathogenic bacteria resistance.

### DESCRIPTION OF RELATED ART

Zasloff, in U.S. Pat. Nos. 5,643,876 and 4,810,777, entitled “Biologically Active Synthetic Magainin Peptides” and “Antimicrobial Compounds,” described a family of synthetic compounds termed “magainin which are capable of inhibiting the growth or proliferation of gram-positive and gram negative bacteria, fungi, virus, and protozoan species.

Haynie, in U.S. Pat. No. 5,847,047, entitled “Antimicrobial Composition of Polymer and a Peptide Forming Amphiphilic Helices of the Magainin-Type,” offers a series of non-natural oligopeptides that share a common amino acid sequence referred to as the core oligopeptide. Such core oligopeptide has antimicrobial effects. The patent also provides N-addition analogues to the core oligopeptide that exhibit higher antimicrobial effects.

Olsen et. al., in U.S. Pat. No. 6,143,498, entitled “Antimicrobial Peptide,” proposed a method of producing human antimicrobial peptides from the defensin superfamily through transformation of host cells. Olsen suggested the production of these defensin-related peptides through transformation of host cells with vectors containing the isolated DNA molecules of the peptides.

Kim, et. al., in U.S. Pat. No. 6,183,992, entitled “Method For Mass Production Of Antimicrobial Peptide,” offered a method of mass producing an antimicrobial peptide. In particular, a fusion gene—containing a basic antimicrovial peptide which ligated directly or indirectly to a negatively charged acidic peptide having at least two cysteine residues—is cloned into an expression vector targeted toward microorganisms such as E. Coli.

All patents and publications are hereby incorporated by reference in their entireties.

### BACKGROUND OF THE INVENTION

Plant diseases caused by bacterial pathogens have had a detrimental effect on global crop production for years. Between 1979 and 1980 India lost up to 60% of its rice crop due to bacterial rice blight. Between 1988 and 1990, there was a 10.1% loss of the global barley crop due to bacterial pathogens, worth $1.9 billion (Baker et al., 1997). In the United States, there was an estimated 44,600 metric ton reduction of soybean crops due to bacterial pathogens in 1994 (Wrath et al., 1996). On the average, pathogens are responsible for a 12-13% reduction of global crop production each year (Dempsey et al., 1998).

A prior effort to combat these devastating pathogens is plant breeding (Mourgues et al., 1998). The results were limited due to the ability of the bacteria to adapt and find a way around the defense mechanism. Agrochemicals have also been used but their application is limited by their toxicity to humans and the environment (Mourgues et al., 1998).

Plant Defense Against Pathogens: Many of the pathways and products in the plant response to phytopathogens have been elucidated with the emergence of molecular biology. The plant defense response can be divided into 3 major categories, early defense (fast), local defense (fast/intermediate) and systemic defense (intermediate to slow) (Mourgues et al., 1998). During the early stage, the plant cell is stimulated by contact with pathogen-produced elicitors. Bacterial genes such as hrp (hypersensitive response and pathogenicity) or avr (avirulence) genes stimulate the plant defense mechanism (Baker et al., 1997). The most prominent early defense response is the HR (hypersensitive response), which leads to cellular death reducing further infection by the pathogen. Local defense entails cell wall reinforcement, stimulation of secondary metabolite pathways, synthesis of thionins and synthesis of PR (pathogenesis-related) proteins (Mourgues et al., 1998). The final phase is known as SAR (systemic acquired resistance), which protects the uninfected regions of the plant.

Engineering Resistance Genetic engineering has allowed for some enhancement of natural defense genes from plants by cloning and over-expression in non-host plants. Cloning of resistance (R) genes has been used to protect rice from bacterial leaf blight (Mourgues et al., 1998). Pathogenesis-related (PR) genes have been cloned from barley and have shown to provide resistance to P. syringae pv. tabaci (Mourgues et al., 1998). Anti-fungal peptides produced by various organisms have been cloned and studied. However, although anti-fungal development has been promising, bacteria still maintain the ability to adapt to plant defenses.

Those skilled in the art will be familiar with antimicrobial peptides. Examples of some of these substances include PGLa (frog skin), defensins (human phagocytes), cecropins (Silkmoth pupae or pig intestine), apidaecins (honeybee lymph), melittin (bee venom), bombinin (toad skin) and the magainins (frog skin). Specifically bactericidal peptides include large polypeptides such as lysozyme (MW 15000 daltons) and attacins (MW 20-23,000 daltons) as well as smaller polypeptides such as cecropin (MW 4000 daltons) and the magainins (MW 2500 daltons). The spectrum of biocidal activity of these peptides is somewhat correlated to size. In general, the large polypeptides are active against limited types and species of microorganisms (e.g., lysozyme against only gram positive bacteria), whereas many of the smaller oligopeptides demonstrate a broad spectrum of antimicrobial activity, killing many species of both gram positive and gram negative bacteria. It has been shown that magainin, cecropins, and bombinin oligopeptides form similar secondary structures described as an amphiphilic helix (Kaiser et al. Annu. Rev. Biophys. Biophys. Chem 16, 561-581, 1987). These peptides with alpha.-helical structures are ubiquitous and found in many organisms. They are believed to participate in the defense against potential microbial pathogens. One of the first biocidal oligopeptides to be isolated from natural sources was bombinin and is described by Csordas et al. (Proc. Int. Symp. Anim. Plant Toxins, 2, 515-523, (1970)). Csordas teaches significant sequence homology between bombinin and melittin, another antimicrobial peptide, isolated from bee venom.

Specifically, the role of magainins from Xenopus laevis (African frog) and its analogues have been investigated by Zasloff et al. (WO 9004408) as pharmaceutical compositions such as a broad-spectrum topical agent, a systemic antibiotic; a wound-healing stimulant; and an anticancer agent (Jacob and Zasloff, 1994). Cuervo et al. (WO 9006129) describe the preparation of deletion analogues of magainin I and II for use as pharmaceutical compositions. They disclose a general scheme for the synthetic preparation of compounds with magainin-like activity and structure. However, the possible agricultural use of magainin-type antimicrobial peptides has not yet been explored. Accordingly, it is an objective of this invention to demonstrate the conference of phytopathogenic bacteria resistance to plants by transforming plant cell plastids to express magainin and its analogues.

Plastid Transformation To date, plastid transformation, particularly has enabled generation of herbicide (Daniell et al., 1998), insect resistant crops (Kota et al., 1999; McBride et al., 1995; DeCosa et al., 2000) and production of pharmaceutical proteins (Guda et al., 2000; Staub et al., 2000). Plastid transformation was selected because of several advantages over nuclear transformation (Daniell, 1999 A, B; Bogorad, 2000; Heifetz, 2000). With concern growing about outcrossing of genetically altered genes, it should be noted that plastid expressed genes are maternally inherited in most crops. Gene containment is possible when foreign genes are engineered via the plastid genome, which prevents pollen transmission in crops that maternally inherit the plastid genome. Because a majority of crop plants inherit their plastid genes maternally, the foreign genes do not escape into the environment. Although pollen from plants that exhibit maternal inheritance contain metabolically active plastids, the plastid DNA is lost during pollen maturation (Helfetz, 2000). Despite the potential advantage of plastid reproduction of AMPs, it was not obvious that AMPs would be produed in this manner. Prior to the patent application there were no published reports of expression of AMPs in plant plastids. Non-obviousness of the disease resistance. Several foreign genes have been expressed within plastids to introduce novel traits including herbicide resistance or insect resistance. However, all of these foreign proteins, without exception, function within plastids. For example, herbicides target proteins or enzymes present within plastids. When engineered plastids are consumed by target insects; insecticidal proteins are released inside the insect gut.

However, in order to use the chloroplast compartment to engineer disease resistance, it was necessary to export foreign proteins into the cytosol where phytopathogens colonize. Therefore, it was not obvious to engineer the plastid genome to confer disease resistance. There are no prior reports or suggestions in the literature that plastid genome could be engineered to confer disease resistance. Also, it is known in the art that antimicrobial peptides are toxic to plant chloroplasts because of the charge on the chloroplast membranes. However, this invention teaches that transgenic plastids expressing antimicrobial peptides rupture at the site of infection upon cell death. Release of large amounts of the antimicrobial peptide prevent the spread of the phytopathogen. Thus, the present invention confirms a novel and unobvious solution to combat phytopathogens that is previously unknown and contrary to all current understanding of chloroplast biology.

Most importantly, small peptides are not stable inside living cells and are highly susceptible to proteolytic degradation. For this reason, small peptides are usually produced as fusion proteins with larger peptides in biological systems. Megainin type peptides are chemically synthesized and never made in biological systems for that reason. Therefore, it was not obvious to express a small peptide of a few amino acids within plastids. Successful expression of this antimicrobial peptide was not anticipated but this invention opens the door for expression of several small peptides within plastids, including hormones.

### SUMMARY OF THE INVENTION

This invention provides a new option in the battle against phytopathogenic bacteria through transformation of the plant plastid genome. The present invention is applicable to all plastids of plants. These include chromoplasts which are present in the fruits, vegetables and flowers; amyloplasts which are present in tubers like the potato; proplastids in roots; leucoplasts and etioplasts, both of which are present in non-green parts of plants. All known methods of transformation can be used to introduce the vectors of this invention into target plant plastids including bombardment, PEG Treatment, Agrobacterium, microinjection, etc.

This invention provides plastid expression constructs which are useful for genetic engineering of plant cells and which provide for enhanced expression of a foreign peptide in plant cell plastids. The transformed plant is preferably a metabolically active plastid, such as the plastids found in green plant tissues including leaves and cotyledons. The plastid is preferably one which is maintained at a high copy number in the plant tissue of interest.

The plastid expression constructs for use in this invention generally include a plastid promoter region and a DNA sequence of interest to be expressed in transformed plastids. The DNA sequence may contain one or a number of consecutive encoding regions, one of which preferably encoding an antimicrobial peptide of the magainin family. Plastid expression construct of this invention is linked to a construct having a DNA sequence encoding a selectable marker which can be expressed in a plant plastid. Expression of the selectable marker allows the identification of plant cells comprising a plastid expressing the marker.

In the preferred embodiment, transformation vectors for transfer of the construct into a plant cell include means for inserting the expression and selection constructs into the plastid genome. This preferably comprises regions of homology to the target plastid genome which flank the constructs.

The plastid vector or constructs of the invention preferably include a plastid expression vector which is capable of importing phytopathogenic bacteria resistance to a target plant species which comprises an expression cassette which is described further herein. Such a vector generally includes a plastid promoter region operative in said plant cells' plastids, a DNA sequence which encode at least an antimicrobial peptide of the magainin family. Preferably, expression of one or more DNA sequences of interest will be in the transformed plastids.

The preferred embodiment of the invention provides a universal plastid vector comprising a DNA construct. The DNA construct includes a 5′ part of a plastid spacer sequence; a promoter, such as Prrn, which is operative in the plastid of the target plant cells; a heterologous DNA sequence encoding at least one antimicrobial peptide of the magainin family; a gene that confers resistance to a selectable marker such as the aadA gene; a transcription termination region functional in the target plant cells; and flanking each side of the expression cassette, flanking DNA sequences which are homologous to a DNA sequence of the target plastid genome, whereby stable integration of the heterologous coding sequence into the plastid genome of the target plant is facilitated through homologous recombination of the flanking sequence with the homologous sequences in the target plastid genome. The vector may further comprise a ribosome binding site (rbs), a 5′ untranslated region (5′UTR). A promoter, such as psbA, accD or 16srRNA, is to be used in conjunction with the 5′UTR. In addition to the encoding region of the antimicrobial peptide, the heterologous DNA sequence of the DNA construct may also include other genes whose expression are desired.

In another embodiment of the invention, non-universal plastid vectors such as pUC, pBlueScript, pGEM may be used as the agent to insert the DNA construct

This invention provides transformed crops, like solanaceous, monocotyledonous and dicotyledonous plants, that are resistant to phytopathogenic bacteria. Preferably, the plants are edible for mammals, including humans. These plants express an antimicrobial peptide at levels high enough to provide upwards of 96% inhibition of growth against Pseudomonas syringae, a major plant pathogen. The transformed plants do not differ morphologically from untransformed plants.

### BRIEF DESCRIPTION OF THE DRAWINGS

FIG. 1. (A) Chloroplast vector used for transformation of Nicotiana tabacum var. Petit Havana. Vector contains the aadA selectable marker gene that confers resistance to spectinomycin, the Prrn promoter, and the TpsbA terminator, (B) Amino acid sequence of the lytic peptide MSI-99.

FIG. 2. (A) Phenotype of T₀ and T₁ transgenic plants. Plants 1-3 are T₀ transgenic plants while plant 4 is untransformed. Plants 5-7 are T₁ transgenic plants. Seedlings germinated on MSO+500 μg/ml spectinomycin (B). Three T₁ transgenic lines (1-3) and Control (4).

FIG. 3. (A) Primers, 8P and 8M used to confirm integration of foreign genes via PCR. 8P anneals with the 5′ end of the aadA gene and 8M anneals with the 3′ end of the 16S rDNA gene. PCR analysis of DNA extracted from T₀ (B), T₁ (C) and T₂ (D) plants run on a 0.8% agarose gel. T₀ (B) Lane 1 1 kb ladder, 2 through 5 transgenic lines, 6 MSI-99 plasmid. T₁ (C) Lane 1, 1 kb ladder, 2 through 4 transgenic, lane 5 plasmid control and lane 6 untransformed plant DNA. T₂ (C) lane 1, 1 kb ladder, 2 through 5 transgenic, lane 6 plasmid control and lane 7 untransformed plant DNA.

FIG. 4. Southern analysis of T₀ and T₁ generations. (A) Probe used to confirm integration of foreign genes. The 2.3 kb probe fragment was cut with BamHI and NotI containing the flanking sequence. (B) Lane 2-6 T.sub.0 transgenic lines, lane 1 untransformed and Lane 7 plasmid DNA. (C) Lanes 2-7 T.sub.1 transgenic lines, Lane 1 untransformed and Lane 8 plasmid DNA.

FIG. 5. In situ bioassays. 5 to 7 mm areas of T₀ transformants and untransformed Petit Havana leaves were scraped with fine grain sandpaper. Ten μl of 8×10⁵, 8×10⁴, 8×10³ and 8×10² cells from an overnight culture of P. syringae were added to each prepared area. Photos were taken 5 days after inoculation FIG. 6. In vitro bioassays for T₀, T₁ and T₂ generations of 3 transgenic lines (10A, 11A and 13A). Five μl of bacterial cells from an overnight culture were diluted to (A₆₀₀ 0.1-0.3) and incubated for 2 hours at 25° C. with 100 μg of total plant protein extract. One ml of LB broth was added to each sample. Samples were incubated overnight at temperature appropriate for the specific bacteria. Absorbance at 600 nm was recorded. Data was analyzed using GraphPad Prism. Negative control was untransformed plant extract. Buffer only was added as a control and stock culture was used as a reference point.

FIG. 7. In vitro bioassays for P. aeruginosa. Five μl of bacterial cells from an overnight culture were diluted to (A₆₀₀ 0.1-0.3) and incubated for 2 hours at 25° C. with 100 μg of total protein extract from T₁ plants. One ml of LB broth was added to each sample. Samples were incubated overnight at 37° C. Absorbance at 600 nm was recorded. Data was analyzed using GraphPad Prism. Negative control was an untransformed plant extract. Buffer only was added as a control and stock culture was used as a reference point.

FIG. 8. Five μl of an overnight culture of P. syringae diluted to (A₆₀₀ 0.1-0.3) was mixed with 100 μg total protein extract from T2 lines 11A and 13A (germinated in the absence of spectinomycin). After 2-hour incubation, 1 ml of LB broth was added to the mixture and incubated over night at 27° C. The following morning absorbance at 600 nm was recorded (A). In parallel, 50 μl of each mix was plated onto LB plates and incubated overnight at 27° C. The next morning a count of viable CFUs were made using the Bio Rad Gell Dock (B).

### DETAILED DESCRIPTION OF THE INVENTION

This invention demonstrates the confering of phytopathogenic resistance in plants through plastid transformation. This invention includes the use of all plastids in plants, including chloroplasts, chloroplasts which are present in fruits, vegetables and flowers, amyloplasts which are present in tubers, proplastids in roots, lencoplasts in non-green parts of plants. In a preferred embodiment of the invention, the chloroplast genome is used. Plastid transformation and expression vectors comprising heterologous DNA encoding magainin and its analogues are provided. The anti-microbial peptide (AMP) used in this invention is an amphipathic alpha-helix molecule that has an affinity for negatively charged phospholipids commonly found in the outer-membrane of bacteria. Upon contact with these membranes, individual peptides aggregate to form pores in the membrane, resulting in bacterial lysis. Because of the concentration dependent action of the AMP, it was expressed via the plastid genome to accomplish high dose delivery at the point of infection. PCR products and Southern blots confirmed plastid integration of the foreign genes and homoplasmy. Growth and development of the transgenic plants was unaffected by expression of the AMP within the plastids. In vitro assays with T₀, T₁ and T₂ plants, confirmed the AMP was expressed at levels high enough to provide 86% (T₀), 88% (T₁) and 96% (T₂) inhibition of growth against Pseudomonas syringae, a major plant pathogen. In situ assays resulted in intense areas of necrosis around the point of infection in control leaves, while transformed leaves showed no signs of necrosis. Even when germinated in the absence of spectinomycin selection, T₂ generation plants showed 96% inhibition of growth against P. syringae.

MSI-99 is an analogue of a naturally occurring peptide (magainin 2) found in the skin of the African frog. Changes have been made to the amino acid sequence to enhance its lytic abilities. Contrary to the prior knowledge in the art which proposed that anti-microbial peptides having high antibacterial activity also have a high potential for toxic activity against the plastid (Everett and Nicholas, 1994), the transgenic plants of this invention grew, flowered and set seeds like the untransformed control.

Key features of cationic peptides such as MSI-99 are a net positive charge, an affinity for negatively charged prokaryotic membrane phospholipids over neutral-charged eukaryotic membranes, and the ability to form aggregates that disrupt the bacterial membrane (Houston et al., 1997; Matsuzaki et al., 1999; Biggin and Sansom, 1999). Given the fact that the outer membrane is an essential and highly conserved part of all bacterial cells, it is highly unlikely that bacteria would be able to adapt (as they have against antibiotics) and to resist the lytic activity of these peptides. In contrast to prokaryotic membranes, the thylakoid membrane consists of primarily glycolipids and galactolipids instead of phospholipids. Monogalactosyldiacylglycerol (MGDG) makes up 50% of membrane lipid and digalactosyldiacylglycerol (DGDG) 30% (Siegenthaler et al., 1998). Both of these lipids are neutral.

An object of this invention is to compartmentalize the expression of the MSI-99 within the plastid. Compartmentalization of lytic enzymes is a natural occurrence in plants. Compartmentalization serves two purposes: to increase the yield of the peptide and to deliver the peptide at the site of the infection. Due to the high copy number associated with plastid expression, a larger amount of the peptide is produced. The higher yield is important due to the concentration-dependent action of the anti-microbial peptide. Further, the peptide would be released at the site of infection during the HR response. When the HR response occurs, cells are lysed. This disrupts the osmotic balance and causes plastids to lyse. This would release the peptide at high concentration resulting in aggregation and formation of pores in the outer membrane of bacteria. This aids in the prevention of the spread of infection by bacteria.

A high level of AMP expression can be expected due to the following reasons. The nature of plastids to move from a somatically unstable heteroplasmic state to a state of homoplasmy itself lends to high expression (Brock and Hagemann, 2000). The A+T % of MSI-99 is 51.39%, which is compatible with the Nicotiana tobacum plastid 61% A+T content (Bogorad et al., 1991; Shimada et al., 1991). Also, published reports from our lab report expression of Cry2A operon (A+T content of 65%) at levels as high as 46% total soluble protein (DeCosa et al., 2000).

MSI-99 was most effective against P. syringae, evidenced by total inhibition of 1000 P. syringae cells with only 1 μg/1000 bacteria (Smith et al. unpublished data). Because the lytic activity of antimicrobial peptides is concentration dependent, the amount of antimicrobial peptide required to kill bacteria was used to estimate the level of expression in transgenic plants. Based on the minimum inhibitory concentration, it was estimated that transgenic plants expressed MSI-99 at 21% of the total soluble protein. Without the availability of antibody for MSI-99, other direct methods of protein estimation were not feasible.

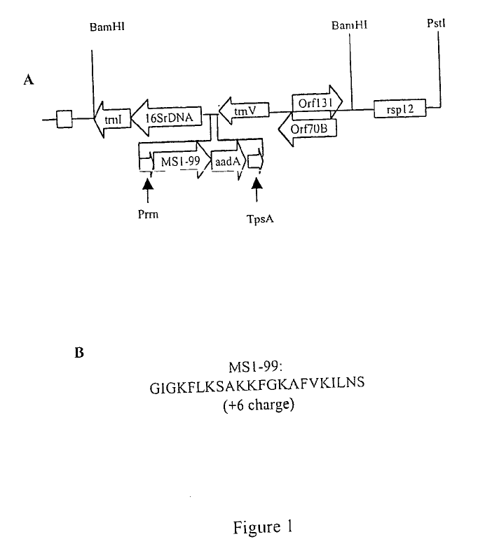

Plastid vectors and plant transformation: The synthetic peptide used in this invention (MSI-99), is an analogue of the naturally occurring 23 amino acid peptide, magainin II. MSI-99 is a 22 amino acid sequence with an overall charge of +6 as shown in FIG. 1. The gene cassette used for transformation consisted of the 16S rRNA promoter, the aadA gene, which confers resistance to spectinomycin, the MSI-99 gene and the psbA (photosynthetic binding protein) terminator. The gene construct may contain, in addition to the MSI-99 gene, another heterologous DNA sequence coding for a gene of interest.

Flanking sequences are from the petunia plastid genome as shown in FIG. 1A. Transformation efficiency was much lower (7%) than that observed using the pLD vector (91%), which contains tobacco homologous flanking sequences. Other vectors that are capable of plastid transformation may be used to deliver the gene cassette into the plastid genome of the target plant cells. Such vectors do include plastid expression vectors such as pUC, pBlueScript, pGEM, and all others identified by Daniell in U.S. Pat. Nos. 5,693,507 and 5,932,479. These publications and patents are herein incorporated by reference to the same extent as if each individual publication or patent was specifically and individually indicated to be incorporated by reference. The vectors preferably include a ribosome binding site (rbs) and a 5′ untranslated region (5′UTR). A promoter operably in green or non-green plastids is to be used in conjunction with the 5′UTR)

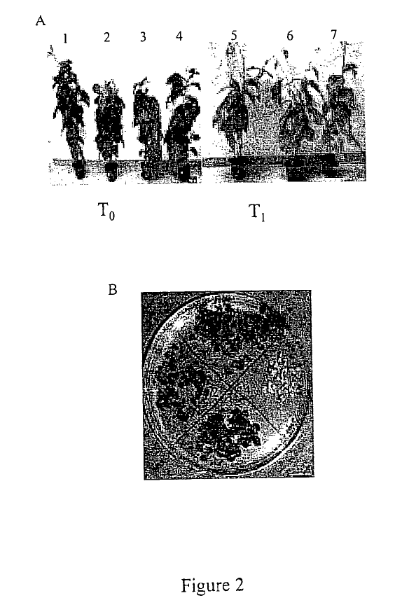

The number of transformants from the total number of shoots determined percent of transformants. Out of 55 spectinomycin resistant shoots screened, only 4 were transformants with the MSI-99 gene and the rest were mutants. All transformants grew healthy with no apparent morphological effects to T₀ and T₁, generations as shown in FIG. 2A. T.sub.1, seeds germinated in the presence of spectinomycin produced healthy green seedlings, while control seedlings were bleached as shown in FIG. 2B.

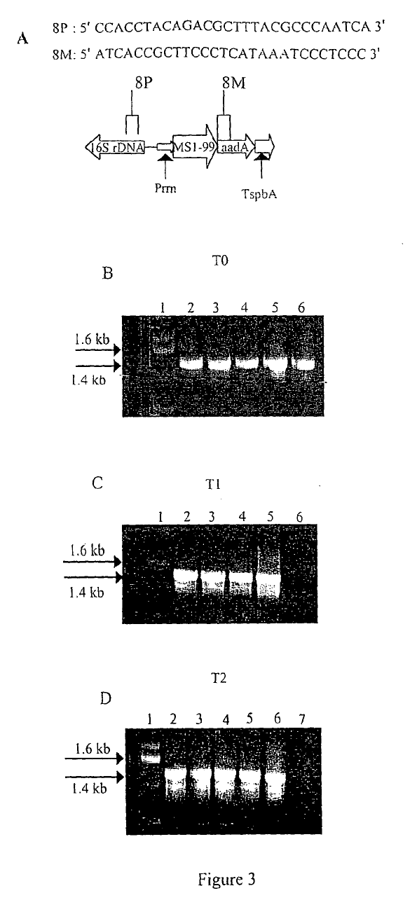

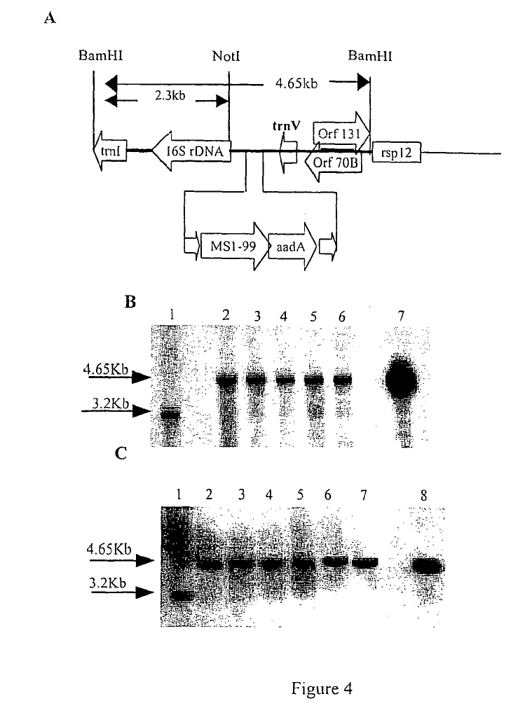

Foreign gene integration, homoplasmy and copy number: PCR was performed by landing one primer on the 5′ end of the aadA coding sequence, not present in native plastid and the 3′ end of the 16S rDNA (FIG. 3A). PCR products of T₀, T₁, and T₂ generations yielded the same size product as the plasmid (MSI-99) as shown in FIG. 3B, C, D confirming integration of the foreign genes. The probe used for the Southern analysis was a 2.3 kb fragment from the 5′end of the tmI (BamHI) to the 3′ end of the 16SrDNA (NotI) (FIG. 4A). The plant DNA was digested with BamHI. DNA from untransformed plants produced a 3.269 kb fragment and transformed plant DNA produced a 4.65 kb fragment. Southern analysis confirmed integration of foreign genes for T₀ and T₁, as shown in FIG. 4B, C. Untransformed DNA showed a 3.2 kb fragment while the transformed contained a 4.65 kb fragment. Presence of some wild type fragments in T₀ transgenic samples indicated some heteroplasmy as shown in FIG. 4B. However, DNA from T₁, generation produced only the 4.65 Kb fragment confirming homoplasmy. As shown in FIG. 4C. A cell is said to be homoplasmic when all of the plastid are uniformly transformed. If only a fraction of the genomes was transformed, the copy number should be less than 10,000 (Bendich, 1987). By confirming that the MSI-99 integrated genome is the only one present in transgenic plants (homoplasmy), one could estimate that the MSI-99 gene copy number could be as many as 10,000 per cell.

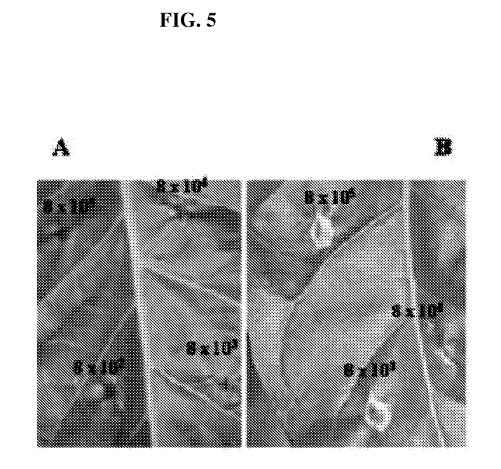

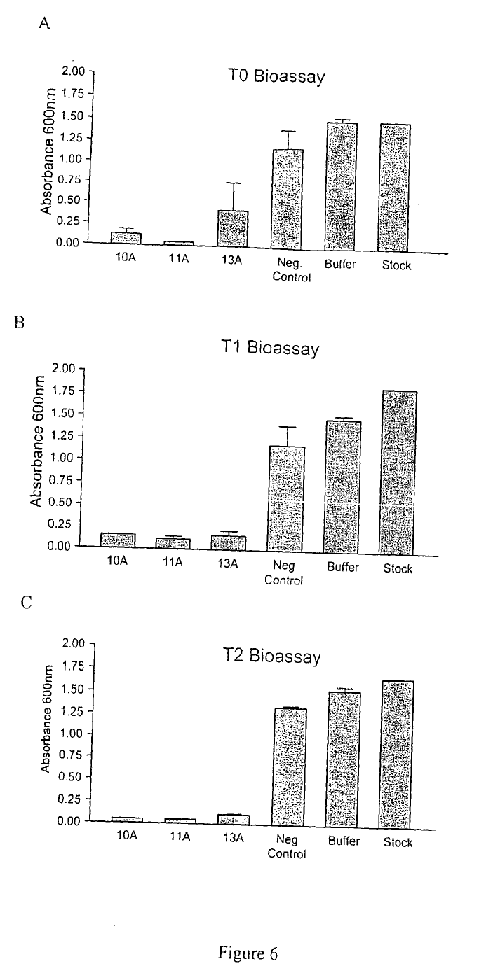

Bioassays: T₀ in situ assays in potted plants (6 to 7 months old) resulted in areas of necrosis surrounding the point of infection in untransformed control, while transgenic leaves showed no areas of necrosis (FIG. 5). Even inoculation of 8×10⁵ cells resulted in no necrosis in transgenic leaves (FIG. 5A), suggesting the local concentration of the antimicrobial peptide to be high. However, plants inoculated with 8×10³ cells displayed intense necrosis as shown in FIG. 5B.

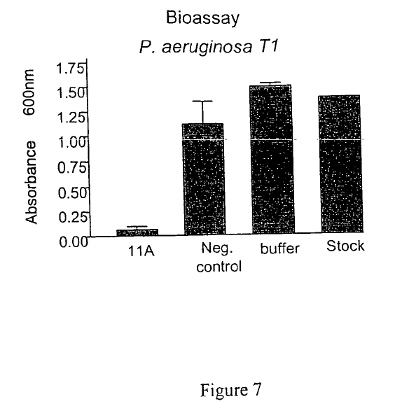

Cell free extracts of T₀, T₁, and T₂ transgenic plants displayed a strong ability to inhibit growth of P. syringae in vitro by 84%, 86% and 96% compared to untransformed plants as shown in FIG. 6. The increase in growth inhibition from T₀ to T₂ can be attributed to heteroplasmy in the T₀ generation that was eliminated in subsequent generations. This indicates the peptides retained their lytic activity and successfully passed on the trait to the subsequent generations. The control had less growth than the buffer only. This is most probably due to natural defense peptides such as defensins and thionins produced by plants (Mourgues et al., 1998). When performing in vitro bioassays against P. aeruginosa, results were similar with T₁, generation showing 96% inhibition of growth (FIG. 7).

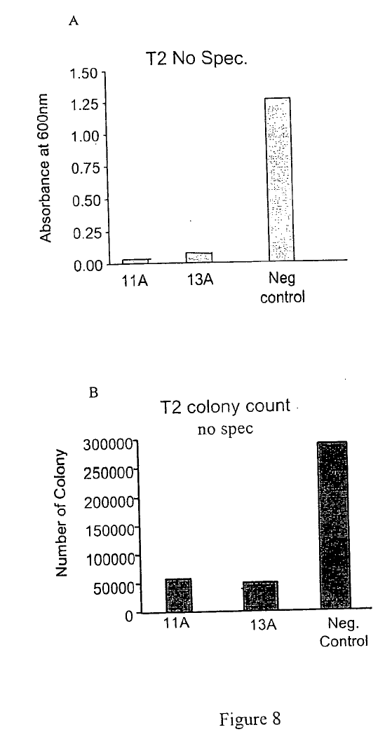

Absorbance readings as shown in FIG. 8A from transgenic plants germinated in the absence of spectinomycin, displayed 96% inhibition of growth that is comparable to transgenic plants germinated in the presence of spectinomycin. Plated cells of bioassay samples from T2 plants germinated in the absence of spectinomycin as shown in FIG. 8B showed 83% inhibition of growth compared to the control. The marginal degree of difference between the plating results and the bioassay results (13%) can be explained by the difference in environment. While the plated bacteria were no longer exposed to active peptides, bacteria in the liquid media were constantly surrounded by active peptides.

Protein Estimation The plate with 10⁻⁵ dilution had 43 CFUs. The equated to 43×10⁶ CFU/ml. The count was adjusted to reflect the 5 μ.l of culture used. This resulted in a count of 21,500 bacterial cells in the initial 5 μl of culture incubated with the peptide. Using 1 μg to kill 1000 P. syringae cells as the reference (Smith et al. unpublished data), the estimated expression of MSI-99 was 21.5 μg in 100 μg soluble protein (21.5%).

The initial low rate of transformation was most likely due to less than 100% homology between the petunia flanking sequences and the tobacco plastid genome. This is not surprising because very low transformation efficiency was also observed when tobacco plastid flanking sequences were used to transform potato plastid genome (Sidorov et al., 1999). Also, other projects in our lab that use the pLD vector (has tobacco flanking sequences) obtained transformation efficiency of 91% transformants to mutants. T.sub.0 and T.sub.1 transgenic plants were healthy and showed no morphological or developmental abnormalities. Retention of lytic activity was evident in the sharp decrease in bacterial growth in the in vitro bioassays (84 to 96%). When comparing Southern blots to lytic activity, lytic activity increased as homoplasmy was reached. Equal lytic activity was also observed in transgenic plants germinated in the absence of spectinomycin (96% inhibition of growth). Transgenic plants transferred to potting soil for 5 to 6 months after being removed from spectinomycin selection, displayed similar antimicrobial properties against inoculations of P. syringae. These observations eliminate the possibility that spectinomycin absorbed into the plant tissue during germination of seeds, may be responsible for the growth inhibition in the in vitro and in situ bioassays. Also, the observation that MSI-99 was equally active in transgenic plants germinated in the presence or absence of spectinomycin shows the stability of the introduced trait in the absence of any selection pressure.

Plastid expression in crops such as tobacco should allow for mass production of the peptide at a lower cost compared to chemical synthesis or production in E. coli. This invention thus demonstrates another option in the on going battle against pathogenic bacteria.

The invention is exemplified by the following non-limiting example.

### EXAMPLE 1

Plant transformation: For plant transformation, Nicotiana tabacum var. Petit Havana seeds were germinated on MSO media at 27° C. with photoperiods of 16 hour light and 8 hour dark. Sterile leaves were bombarded using the Bio-Rad Helium driven PDS-1000/He System. After bombardment, leaves were wrapped and kept in the dark for 48 hours. Leaves were then cut into 1 cm² squares and placed on a petri dish containing RMOP media with 500 μg/ml spectinomycin (first round of selection). Four to six weeks later, shoots were transferred to fresh media and antibiotic (second round of selection). Shoots that appeared during the second selection were transferred to bottles containing MSO and spectinomycin (500 μg/ml). Plants were screened via PCR for transformation. Those that were PCR positive for the presence of the MSI-99 gene were transferred to pots and grown in chambers at 27° C. with photoperiods of 16-hour light and 8-hour dark. After flowering, seeds were harvested and sterilized with a solution of 1-part bleach and 2-part water with 1 drop of tween-20. Seeds were vortexed for 5 minutes then washed 6 times with 500 μl of dH₂O and dried in speed vac. T₁, and T₂ seeds were germinated on MSO+500 μg/ml spectinomycin. Untransformed Petit Havana seeds were germinated on the same media as a control to ensure the spectinomycin was active.

PCR conformation Plant DNA extraction on T₀, T₁, and T₂ was performed using the QIAGEN DNeasy Mini Kit on putative transgenic samples and untransformed plants. PCR primers were designed using Primer Premier software and made by GIBCO BRL. Primer (8p:5′ATCACCGCTTCCCTCATAAATCCCTCCC3′) anneals with the 5′ end of the aadA and primer (8M:5′CCACCTACAGACGCTTTACGCCCAATCA3′) anneals with the 3′ end of 16SrDNA as shown in FIG. 3. PCR was carried out using the Gene Amp PCR system 2400 (Perkin-Elmer). Samples were run for 29 cycles with the following sequence: 94° C. for 1 minute, 65° C. for 1 minute and 72° C. for 3 minutes. The cycles were proceeded by a 94° C. denaturation period and followed by a 72° C. final extension period. A 4° C. hold followed the cycles. PCR products were separated on agarose gels.

Southern analysis: Integration of foreign genes for T₀ and T₁, was determined by Southern blot analysis. DNA from transformed and untransformed plants was digested with BamHI and run on a 0.7% agarose gel. The DNA was then transferred to a nylon membrane by capillary action. The probe was digested with BamHI and NotI and was labeled with 32 P using the Probe Quant™ G-50 Micro Colums and protocol (Amersham). Labeled probe was hybridized with the nylon membrane using the Stratagene QUICK-HYB hybridization solution and protocol. Membrane was exposed to film, and developed.

In vitro bioassay: P. syringae and P. aeruginosa were cultured overnight prior to the assay. 50 mg of leaf tissue (minus mid-rib) was grounded in a micro-centrifuge containing 150 μl of phosphate buffer pH5.5 with 5 mM PMSF and 5 mM with a plastic pestle. Samples were centrifuged for 5 minutes at 10,000×g at 4° C. Supernatant was transferred to a fresh tube and kept on ice. Protein concentration was determined by Bradford assay. One hundred .mu.g of total plant protein was mixed with 5 μl of bacteria from overnight culture in a falcon tube. Initial absorbency ranged from 0.1 to 0.3 (A₆₀₀). Tubes were incubated for 2 hours at 25° C. on a rotary shaker at 125 rpm. One ml of LB broth was added and tubes were allowed to incubate for 18 hours at 27° C. for P. syringae and 37° C. for P. aeruginosa on a rotary shaker at 125 rpm. Absorbance (A.sub.600) was read for each tube. Results were statistically analyzed using GraphPad Prism.

T₀ rule out spectinomycin as the cause of growth inhibition, the same experiment with P. syringae was repeated using T₂ plants that were geminated on MSO with no spectinomycin. For confirmation of the absorption readings, a serial dilution was made of samples after the initial 2-hour incubation. Dilutions of 10⁻³ to 10⁻⁵ were plated onto LB plates and incubated overnight at 27° C. The next morning a count of viable CFUs were made using the Bio Rad Gell Dock.

To estimate the level of protein expression, a serial dilution was prepared from the starting bacterial culture (Absorbance₆₀₀, 0.1-0.3) used for the In vitro bioassay. Fifty μl of each dilution was plated on LB medium and incubated overnight at 27° C. The following morning, CFUs were counted using the Bio Rad Gel Dock and the amount of cells used in the bioassay was calculated. The minimum inhibitory concentration of 1 .mu.g/1000 P. syringae cells was used to determine antimicrobial peptide concentration in 100.mu.g of cell free plant extracts.

In situ bioassay: P. syringae was cultured overnight prior to the assay. Five to seven mm areas of T₀ transformants and untransformed Petit Havana leaves were scraped with fine grain sandpaper. Ten μl of 8×10⁵, 8×10⁴, 8×103 and 8×10² cells from an overnight culture of P. syringae were added to each prepared area. Photos were taken 5 days after inoculation.

### REFERENCES

Baker B, Zambryski P, Staskawicz S P, Dinesh-Kumar. (1997) Signaling in Plant-Microbe Interactions. Science. 276: 726-723
Bendich A. J. (1987) Why do chloroplasts and mitochondria contain so many copies in their genome? BioEssays. 6: 279-282.
Biggin P, Sansom M. (1999) Interactions of (x-helices with lipid bilayers: a review of simulation studies. Biophysical Chemistry. 76: 161-183
Bock R, Hagemann R. (2000) Extracellular inheritance: Plastid genomics: Manipulation of plastid genornes and biotechnological applications. Progress in Botany. 6: 76-90
Bogorad L. (2000) Engineering chloroplasts: an alternative site for foreign genes, proteins, reactions and products. TIBTECH 18: 257-263
Bogarad L, Vasil 1. (1991) The Molecular Biology of Plastids. San Diego: Academic Press, 38
Cary J, Rajasekaran K, Jaynes J, Cleveland T. (2000) Transgenic expression of a gene encoding a synthetic antimicrobial peptide results in inhibition of fungal growth in vitro and in planta. Plant Science. 154: 171-181
Daniell H. (1997) Transformation and foreign expression in plants mediated by microprojectile bombardment. Methods in Molecular Biology. 62: 463-489
Daniell H. (1999 A) New tools for plastid genetic engineering. Nat. Blotechnol. 17:
Daniell H. (1999 B) GM crops: Public perception and scientific solutions. Trends in plant science. 4: 467-469
Daniell H, Datta R, Varma S, Gray S, Lee S. B. (1998) Containment of herbicide resistance through genetic engineering of the chloroplast genome. Nat. Blotechnol. 16: 345-348
DeCosa B, R. Moar W, Lee S. B, Miller M, Daniell H (2000) HyperExpression of the Cry2Aa2 operon in chloroplasts leads to the formation of insecticidal crystals. Nat. Blotechnol. In press
Everett, Nicholas. (1994) Design of Antifungal peptides for agricultural applications. Ed. Hedin, Paul., Merin, Julius, & Hollingworth, Robert. Washington D.C.: American Chemical Society, 278-292
Guda C, Lee S. B., Daniell H. (2000) Stable expression of a biodegradable protein-based polymer in tobacco chloroplasts. Plant Cell Reports. 19: 257-262
Heifetz P. (2000) Genetic engineering of the plastid. Blochimie. 82: 655-666
Houston Jr M. E., Kondejewski L, Gough M, Fidai S, Hodges R. S, Hancock R. (1997) Influence of performed .alpha.-helix and .alpha.-helix induction on the activity of cationic antimicrobial peptides. J. Peptide Res. 52: 81-88
Jacob L, Zasloff M. (1994) Potential therapeutic applications of magainins and other antimicrobial; agents of animal origin. Antimicrobial peptides. (Ciba Foundation Symposium 186): 197-223
Kota M, Daniell H, Varma S, Garczynski F, Gould F, Moar W J. (1999) Over expression of the Bacillus thuringiensis Cry2A protein in plastids confers resistance to plants against susceptible and Btresistant insects. Proc. Natl. Acad. Sci. USA.
Matsuzaki K. (1998) Magainins as paradigm for the mode of action of pore forming polypeptides. Biochimica et Blophysica Acta. 1376: 391400
McBride K. E, Svab Z, Schaaf D. J, Hogen P. S, Stalker D. M, Maliga P. (I 995) Amplification of a chimeric Bacillus gene in chloroplasts leads to extraordinary level of an insecticidal protein in tobacco. Blo/technology 13: 362-365
Mourgues F, Brisset M. N, Cheveau E. (1998) Strategies to improve plant resistance to bacterial diseases through genetic engineering. Trends in Biotechnology. 16: 203
Neuhas J, Sticher L, Meins, Jr F, Boller T. (1991) A short C-terminal sequence is necessary and sufficient for the targeting of chitinases to the plant vacuole. Proc. Natl. Acad. Sci. USA. 88: 10362-10366
Shimada H, Sugiura M. (1991) Fine structural features of the plastid genome: comparison of the sequenced chloroplast genomes. Nucleic Acids Research 19: 983-995
Sidorov V, Kasten D, Pang S, Hajdukiewicz P, Saub J, Nehra N. (1999) Stable chloroplast transformation in potato: use of green fluorescent protein as a plastid marker. The Plant Journal. 19: 209-216
Siegenthaler Paul-Andr6, Murata Noria. Ed. (1998) Lipids in Photosynthesis: Structure, Function and Genetics. Boston: Kluwer, 1-52, 119-144.
Staub J, Garcia B, Graves J, Hajdukiewicz P, Hunter P, Nehra N, Paradkar V, Schlittler M, Carroll J, Saptola L, Ward D, Ye G, Russell D. (2000) High-yield production of a human therapeutic protein in tobacco chloroplasts. Nat. Biotechnol. 18: 333-338.
Tumnieler B, Kiewitz C. (1999) Cystic Fibrosis: an inherited ibility to bacterial infections. Molecular Medicine Today. 5: 351-358
Utsugi T, Schroit A, Connor J, Bucana C, Fidler L. (1991) Elevated expression of phosphatidylserine in the outer leaflet of human tumor cells and recognition by activated human blood monocytes. Cancer Res. 51: 3062-3066
Wrath J. A, Anderson T. R, Arsyad D. M, Gai J, Ploper L. D, Portapuglia A, Ram H. H, Yorinori J T. (1996) Soybean Disease Loss Estimates for the Top 10 Soybean Producing Counties in 1994. Plant disease. 81: 107-110

The teachings of all references cited herein are incorporated in their entirety to the extent they are not inconsistent with the teachings herein. This invention provides a novel method to confer disease resistance to plants. Plant plastids are transformed using a plastid vector which contains heterologous DNA sequences coding for a cytotoxic antimicrobial peptide. Transgenic plants are capable of fighting off phytopathogenic bacterial infection.

1. A stable plastid transformation and expression vector which comprises an expression cassette comprising, as operably linked components in the 5′ to the 3′ direction of translation, a promoter operative in said plastid, a selectable marker sequence, a heterologous DNA sequence coding for cytotoxic antimicrobial peptide (AMP), transcription termination functional in said plastid, and flanking each side of the expression cassette, flanking DNA sequences which are homologous to a DNA sequence of the target plastid genome, whereby stable integration of the heterologous coding sequence into the plastid genome of the target plant is facilitated through homologous recombination of the flanking sequence with the homologous sequences in the target plastid genome.

2. A vector of claim 1, wherein the plastid is selected from the group consisting of chloroplasts, chromoplasts, amyloplasts, proplastide, leucoplasts and etioplasts.

3. A vector of claim 1, wherein the antimicrobial peptide is selected from the groups of defensins, PGLA (frog skin), cecropins, apidaecins, melittin, bombinin and magainin.

4. A vector of claim 3, wherein the antimicrobial peptide is magainin I or II.

5. A vector of claim 1, wherein the selectable marker sequence is an antibiotic-free selectable marker.

6. An integration and expression vector of claim 1 competent for stably transforming a plastid genome of different plant species wherein the flanking DNA sequences are homologous to a spacer sequence of the target plastid genome and the sequence is conserved in the plastid genome of different plant species.

7. A stably transformed plant which comprises plastid stably transformed with the vector of claim 1, or the progeny thereof, including seeds.

8. A stably transformed plant of claim 7 which is a solanaceous plant.

9. A stably transformed plant of claim 7 which is a monocotyledonous or dicotyledonous plant.

10. A stably transformed plant of claim 9 which is maize, rice, grass, rye, barley, oat, wheat, soybean, peanut, grape, potato, sweet potato, pea, canola, tobacco, tomato or cotton.

11. A stably transformed plant of claim 7 which is edible for mammals and humans.

12. A stably transformed plant of claim 7 in which all the chloroplasts are uniformly transformed.

13. A stably transformed plant of claim 7 in which the transformed plastid of the plants including subsequent generations are capable of enhanced levels of expression.

14. A stably transformed plant of claim 7 in which transgenic plants germinated in the absence of antibiotic selectable marker sequence, like spectinomycin.

15. A method for stably transforming a target plant to control a phytopathogenic bacteria which comprises introducing an integration and expression vector of claim 1, into a plastid genome of the target plant, and allowing the transformed plant to grow.

16. A vector of claim 1 wherein the antimicrobial peptide is a cationic amphiphipathic alpha-helix molecule which has affinity for negatively charged phospholipides in the outer membrane of the target bacteria and which is functional to form aggregates that disrupt and lyse the bacterial membrane of the target microbe, and in the prevention of the spread of infection by the bacteria.

17. A vector of any one of claim 1, wherein said vector further comprises a ribosome binding site (rbs) and a 5′ untranslated region (5′UTR).

18. A method of claim 15, wherein said vector further comprises a ribosome binding site (rbs) and a 5′ untranslated region (5′UTR).

19. A plastid transformation and expression vector for stably transforming a plastid, wherein the vector comprises an expression cassette comprising, as operably linked components: a plastid promoter which is operative in said plastid, a selectable marker sequence, a heterologous DNA coding sequence for a cytotoxic antimicrobial magainin family peptide (AMP) or a magainin analog, a transcription termination sequence functional in said plastid, and flanking each side of the expression cassette, a flanking DNA sequence which is homologous to a DNA sequence of a plastid genome, whereby stable integration of the heterologous DNA coding sequence into the plastid genome of a target plant's cell is facilitated through homologous recombination of the flanking DNA sequence with a homologous sequence in the plastid genome.


SEQUENCE LISTING (3 sequences)

SEQ ID NO: 1 - DNA, 28 bp, Artificial Sequence
  Description of Artificial Sequence Primer
atcaccgcttccctcataaatccctccc

SEQ ID NO: 2 - DNA, 28 bp, Artificial Sequence
  Description of Artificial Sequence Primer
ccacctacagacgctttacgcccaatca

SEQ ID NO: 3 - protein, 22 aa, Artificial Sequence
  Description of Artificial Sequence Synthetic peptide
GIGKFLKSAKKFGKAFVKILNS